(19) United States
(12) Patent Application Publication
(10) Pub. No.: US 2025/0381190 A1
(43) Pub. Date: Dec. 18, 2025

(54) THERAPEUTIC COMBINATIONS OF CAPIVASERTIB AND VENETOCLAX

(71) Applicant: AstraZeneca AB, Södertälje (SE)

(72) Inventors: Simon Thomas BARRY, Cambridge (GB); Hannah DRY, Wilmington, DE (US); Jerome METTETAL, Wilmington, DE (US); Brandon WILLIS, Wilmington, DE (US)

(21) Appl. No.: 19/127,968

(22) PCT Filed: Nov. 15, 2022
(86) PCT No.: PCT/EP2022/081881

§ 371 Date: May 7, 2025

(51) Int. Cl. A61K 31/519; A61K 31/635; A61P 35/00

(52) U.S. Cl. CPC A61K 31/519; A61K 31/635; A61P 35/00

(57) ABSTRACT

Therapeutic combinations of capivasertib and venetoclax are described. The combinations can be useful in the treatment of B-cell malignancies. In some embodiments, reduced dosing (e.g., reduced frequency and/or reduced amount) of venetoclax can mitigate or abrogate effects such as body weight loss that may be experienced with an initial dosage regime.

### BACKGROUND

B-cell malignancies represent a class of leukemias and lymphomas arising from dysregulated growth of B-cells. Although understanding of the biology and genetics of such diseases has increased in recent years, there remains a high unmet need for treatments addressing this class of cancers.

AKT is a serine/threonine-specific protein kinase that plays a key role in multiple cellular processes such as glucose metabolism, apoptosis, cell proliferation, transcription, and cell migration. Mammalian cells express three closely related AKT isoforms that are encoded by different genes: AKT1, AKT2, and AKT3. Capivasertib, having the structure

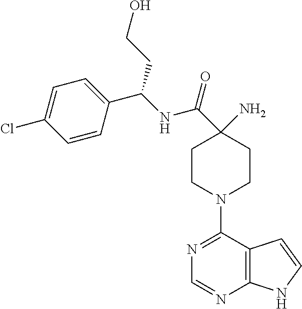 [SMILES: NC1(C(=O)N[C@@H](CCO)c2ccc(Cl)cc2)CCN(c2ncnc3[nH]ccc23)CC1]

also known as AZD5363 or its chemical name (S)-4-amino-N-(1-(4-chlorophenyl)-3-hydroxypropyl)-1-(7H-pyrrolo[2,3-d]pyrimidin-4-yl)piperidine-4-carboxamide) is a selective inhibitor of all three AKT isoforms. Capivasertib is described in, e.g., WO 2009/047563 (which is incorporated herein by reference), and is being evaluated in clinical studies for use in treating cancers including breast cancer and prostate cancer.

Anti-apoptotic Bcl-2 proteins are associated with a number of diseases including B-cell malignancies. Overexpression of Bcl-2 proteins correlates with resistance to chemotherapy, clinical outcome, disease progression, overall prognosis or a combination thereof in various cancers and disorders of the immune system. Venetoclax, having the structure

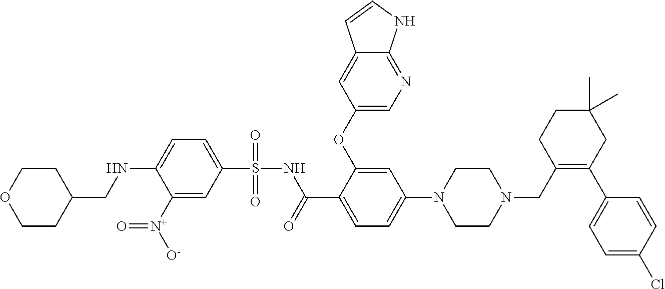 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

also referred to as ABT-199 or its chemical name 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) is a selective Bcl-2 inhibitor, described in, e.g., U.S. Pat. Nos. 8,546,399 and 9,174,982 (each of which is incorporated herein by reference). It is approved for treatment of chronic lymphocytic leukemia, small lymphocytic leukemia, and acute myeloid leukemia.

### SUMMARY

In one aspect, there is provided a method of treating a B-cell malignancy in a patient in need thereof, comprising: administering to the patient a first amount of capivasertib or a pharmaceutically acceptable salt thereof, and a second amount of venetoclax or a pharmaceutically acceptable salt thereof; wherein the first amount and second amount together comprise a therapeutically effective amount.

In one aspect, there is provided a method of treating a B-cell malignancy in a patient in need thereof, comprising:
in a seven-day dosage cycle, administering to the patient:
a first amount of capivasertib or a pharmaceutically acceptable salt thereof on two days or on four days; and
a second amount of venetoclax or a pharmaceutically acceptable salt thereof on seven days; and
in a later seven-day dosage cycle, administering to the patient:
a first amount of capivasertib or a pharmaceutically acceptable salt thereof on two days or on four days; and
a second amount of venetoclax or a pharmaceutically acceptable salt thereof on one, two, three, four, five or six days;
wherein the first amount and second amount together comprise a therapeutically effective amount.

In one aspect, there is provided a method of treating a B-cell malignancy in a patient in need thereof, comprising:
in a seven-day dosage cycle, administering to the patient:
from 300 to 800 mg of capivasertib twice daily on days one, two, and optionally days three and four; and
from 20 to 400 mg of venetoclax once daily on seven days;
in a later seven-day dosage cycle, administering to the patient:
from 300 to 800 mg of capivasertib twice daily on days one, two, and optionally days three and four of the later seven-day dosage cycle; and
from 20 to 400 mg of venetoclax once daily on day one and on an additional one, two, or three days of the later seven-day dosage cycle.

In one aspect, there is provided capivasertib for use in any one of the methods described herein.

In one aspect, there is provided venetoclax for use in any one of the methods described herein.

In one aspect, there is provided a kit comprising (i) capivasertib, and (ii) instructions for the use of capivasertib in any one of the methods described herein.

In one aspect, there is provided a combination of capivasertib and venetoclax for use in any one of the methods described herein, wherein the combination is for the simultaneous, separate, or sequential dosing of capivasertib and venetoclax.

In one aspect, there is provided a use of a combination of capivasertib and venetoclax in any one of the methods described herein, the combination comprising: a first amount of capivasertib or a pharmaceutically acceptable salt thereof, and a second amount of venetoclax or a pharmaceutically acceptable salt thereof; wherein the first amount and second amount together comprise a therapeutically effective amount; wherein the combination is for the simultaneous, separate, or sequential dosing of capivasertib and venetoclax.

Other features, objects, and advantages will be apparent from the description and drawings, and from the claims.

### BRIEF DESCRIPTION OF THE DRAWINGS

FIG. 1 shows body weight change following a dosing schedule of capivasertib 130 mg/kg BID 10/14 4 days on/3 days off; venetoclax 100 mg/kg QD in AM (15 min after capivasertib).

FIG. 2 shows capivasertib plasma levels following a dosing schedule of capivasertib 130 mg/kg BID 10/14 4 days on/3 days off; venetoclax 100 mg/kg QD in AM (15 min after capivasertib).

FIG. 3 shows venetoclax plasma levels following a dosing schedule of capivasertib 130 mg/kg BID 10/14 4 days on/3 days off; venetoclax 100 mg/kg QD in AM (15 min after capivasertib).

FIG. 4 shows body weight change following a dosing schedule of capivasertib 130 mg/kg BID 10/14 4 days on/3 days off; venetoclax 100 mg/kg QD in AM (4 hours after capivasertib).

FIG. 5 shows capivasertib plasma levels following a dosing schedule of capivasertib 130 mg/kg BID 10/14 4 days on/3 days off; venetoclax 100 mg/kg QD in AM (4 hours after capivasertib).

FIG. 6 shows venetoclax plasma levels following a dosing schedule of capivasertib 130 mg/kg BID 10/14 4 days on/3 days off; venetoclax 100 mg/kg QD in AM (4 hours after capivasertib).

FIG. 7 shows body weight change following a prolonged dosing schedule of capivasertib 130 mg/kg BID 10/14 4 days on/3 days off; venetoclax 100 mg/kg QD in AM (4 hours after capivasertib).

FIG. 8 shows venetoclax plasma concentration following dosing with venetoclax 100 mg/kg QD monotherapy, and capivasertib 130 mg/kg BID 10/14 4 days on/3 days off; venetoclax 100 mg/kg QD in AM (4 hours after capivasertib).

FIG. 9 shows body weight change following prolonged dosing with capivasertib 130 mg/kg BID 10/14 4 days on/3days off; venetoclax 100 mg/kg QD in AM (+4 h) 4 days on/3 days off.

FIG. 10 shows the tumor volume over time for capivasertib 130 mg/kg BID 10/14 4 days on/3days off; venetoclax 100 mg/kg QD in AM (4 hours after capivasertib) 4 days on/3 days off vs vehicles and individual drugs dosed on the same schedule.

FIG. 11 shows body weight change following prolonged dosing with capivasertib 130 mg/kg BID 10/14 4 days on/3days off; venetoclax 100 mg/kg QD 4 days off/3 days on.

FIG. 12 shows the tumor volume over time for capivasertib 130 mg/kg BID 10/14 4 days on/3days off; venetoclax 100 mg/kg QD 4 days off/3 days on vs vehicles and individual drugs dosed on the same schedule.

FIG. 13 shows body weight change following prolonged dosing with capivasertib 130 mg/kg BID 10/14 4 days on/3days off; venetoclax 100 mg/kg QW or 2QW.

FIG. 14 shows the tumor volume over time for capivasertib 130 mg/kg BID 10/14 4 days on/3days off; 100 mg/kg venetoclax QW or 2QW vs vehicles and individual drugs dosed on the same schedule.

FIG. 15 shows the tumor volume over time for capivasertib 45 mg/kg BID 10/14 4 days on/3days off; venetoclax 100 mg/kg QD 5 days on/2 days off (AM, 15 min after capivasertib) vs vehicle.

FIG. 16 shows body weight change following dosing with capivasertib 45 mg/kg BID 10/14 4 days on/3days off; venetoclax 100 mg/kg QD 5 days on/2 days off (AM, 15 min after capivasertib).

FIG. 17 shows the tumor volume over time for capivasertib 130 mg/kg BID 10/14 4 days on/3days off; venetoclax 30 mg/kg QD 5 days on/2 days off (AM, 15 min after capivasertib) vs vehicle.

FIG. 18 shows body weight change following dosing with capivasertib 130 mg/kg BID 10/14 4 days on/3days off; venetoclax 30 mg/kg QD 5 days on/2 days off (AM, 15 min after capivasertib).

### DETAILED DESCRIPTION

Described herein are methods of treating a B-cell malignancy in a patient in need thereof. The methods can include administering both capivasertib (or a pharmaceutically acceptable salt thereof) and venetoclax (or a pharmaceutically acceptable salt thereof). The methods can include administering both capivasertib and venetoclax in respective dosage amounts and on respective dosage schedules so as to provide a therapeutic effect. Advantageously, the combinations described may provide therapeutic effect while minimising the potential for side effects such as body weight loss.

Some B-cell malignancies that can be treated by the combinations of capivasertib and venetoclax described herein include: diffuse large B-cell lymphoma (DLBCL), germinal center B-cell-like diffuse large B-cell lymphoma (GCB-DLBCL), chronic lymphocytic leukemia (CLL), small lymphocytic lymphoma (SLL), or follicular lymphoma.

The term “combination” as used herein refers to simultaneous, separate, or sequential administration of two or more agents. In one aspect, “combination” can refer to simultaneous administration (e.g., administration of both agents in a single dosage form). In another aspect, “combination” refers to separate administration (e.g., administration of both agents in separate dosage forms, but at substantially the same time). In a further aspect, “combination” refers to sequential administration (e.g., where a first agent is administered, followed by a delay, followed by administration of a second or further agent).

The term “dosage cycle” as used herein refers to a repeating unit of dosage schedule. For example, a dosage cycle may repeat every seven days, every 10 days, every 14 days, or every 28 days, or other length of time. The terms “dosage schedule” or “dosing schedule” as used herein refer to the schedule, i.e., times and intervals at which a given drug is dosed. A dosage schedule can be continuous or intermittent.

An intermittent dosage schedule can include dosage holidays, i.e., periods of time during which an agent is not dosed. For the purposes of illustration, in a seven-day dosage cycle, an intermittently dosed agent might be given on days one and two, but not given on days three, four, five, six, or seven. The dosage cycle would then repeat. This illustration could be referred to as a 2 on/5 off schedule, where the agent is given for two days followed by a five day holiday, and then repeated every seven days. Other variations are possible, e.g., a 2 on/5 off schedule where the two days of dosing are non-consecutive.

A continuous dosing schedule, in contrast, includes no holidays during a dosage cycle. Thus, for illustration, in a in a seven-day dosage cycle, a continuously dosed agent would be given on days one, two, three, four, five, six, and seven. The dosage cycle would then repeat.

When a subject is given more than one agent, the two agents can be dosed on the same or different dosage schedules. For example, both a first agent and a second agent can be dosed continuously; or the first agent continuously and the second intermittently (or vice-versa); or both the first and second agents can be dosed on an intermittent schedule. If both agents are on an intermittent schedule, the schedules can be the same or different. In instances where two (or more) agents are given on the same day, they can be given simultaneously, separately, or sequentially.

When two (or more) agents are given in combination, they may each be given at the same dose amount and on the same schedule as when given as monotherapy (i.e., as a single agent, not in combination with other agent(s)). In other cases, the dosage amount and/or dose schedule of one or more agents can be altered from the amount and schedule used in monotherapy.

When capivasertib and venetoclax are given as a combination, they can act synergistically. For example, both capivasertib and venetoclax have activity against DLBCL cell lines when used individually. In vitro, their effect is greater than additive when used in combination.

When capivasertib and venetoclax are given as a combination using their respective monotherapy dosage amounts and dosage schedules, mouse models have shown the potential for body weight loss. For example, mice bearing xenograft tumors (e.g., DLBCL xenograft tumors) undergo body weight loss (BWL) upon treatment with capivasertib and venetoclax at their respective monotherapy doses and with a short (e.g. 15 minutes) delay between respective doses.

Surprisingly, such body weight loss can be mitigated or abrogated by altering the dosage amount and/or schedule of venetoclax. Furthermore, the body weight loss can be mitigated or abrogated without altering the dosage amount and/or schedule of capivasertib. Further still, the body weight loss can be mitigated or abrogated while maintaining efficacy.

When given as monotherapy, capivasertib can be dosed intermittently, e.g., on four days of a seven-day dosage cycle. For example, from 50 to 900 mg of capivasertib can be dosed twice daily on days one, two, three and four of the first seven-day dosage cycle. In some embodiments, from 300 to 700 mg of capivasertib can be dosed twice daily on days one, two, three and four of the first seven-day dosage cycle. For example, from 50 to 900 mg of capivasertib can be dosed twice daily on days one, and two of the first seven-day dosage cycle. In some embodiments, from 300 to 700 mg of capivasertib can be dosed twice daily on days one, two, three and four of the first seven-day dosage cycle.

In some embodiments, the dosing cycle is 7 days, but there is a drug holiday during the fourth cycle (i.e., capivasertib is not given during the final week of a 4-week period). Thus, in some embodiments, capivasertib is given 4 days on, 3 days off for three weeks, and not given during week four; in other words, capivasertib is given on days one, two, three, four, eight, nine, ten, eleven, fifteen, sixteen, seventeen, and eighteen of a 28-day dosing cycle. In some embodiments, capivasertib is given 2 days on, 5 days off for three weeks, and not given during week four; in other words, capivasertib is given on days one, two, eight, nine, fifteen, and sixteen of a 28-day dosing cycle. Other dosage schedules and dosage amounts of capivasertib can be used.

In some embodiments, capivasertib is administered to the subject on an intermittent dosage schedule. Administering capivasertib on an intermittent dosage schedule can, for example, have greater effectiveness and/or tolerability than on a continuous dosing schedule. In one aspect, capivasertib is intermittently dosed on a 1 day on/6 days off schedule (i.e., capivasertib is administered for one day followed by a six-day holiday). In another aspect, capivasertib is intermittently dosed on a 2 days on/5 days off schedule (i.e., capivasertib is administered for two days followed by a five-day holiday). In another aspect, capivasertib is intermittently dosed on a 3 days on/4 days off schedule (i.e., capivasertib is administered for three days followed by a four-day holiday). In another aspect, capivasertib is intermittently dosed on a 4 days on/3 days off schedule (i.e., capivasertib is administered for four days followed by a three-day holiday). In another aspect, capivasertib is intermittently dosed on a 5 days on/2 days off schedule (i.e., capivasertib is administered for five days followed by a two-day holiday). In another aspect, capivasertib is intermittently dosed on a 6 days on/1 day off schedule (i.e., capivasertib is administered for six days followed by a one-day holiday).

The dosing cycle of such embodiments would then repeat as long as tolerable and beneficial for the subject. In some embodiments, the dosing cycle is 7 days. In some embodiments, the dosing cycle is 28 days.

In some embodiments, capivasertib is administered twice daily (BID) under an intermittent dosing schedule. In one aspect, capivasertib is administered under an intermittent dosing schedule at a dosage from about 50 mg to about 900 mg twice daily. In another aspect, capivasertib is administered under an intermittent dosing schedule at a dosage from about 150 mg to about 750 mg twice daily. In another aspect, capivasertib is administered under an intermittent dosing schedule at a dosage from about 200 mg to about 700 mg twice daily. In another aspect, capivasertib is administered under an intermittent dosing schedule at a dosage from about 225 mg to about 675 mg twice daily. In another aspect, capivasertib is administered under an intermittent dosing schedule at a dosage from about 250 mg to about 650 mg twice daily. In another aspect, capivasertib is administered under an intermittent dosing schedule at a dosage from about 300 mg to about 625 mg twice daily. In another aspect, capivasertib is administered under an intermittent dosing schedule at a dosage from about 200 mg to about 300 mg twice daily. In another aspect, capivasertib is administered under an intermittent dosing schedule at a dosage from about 300 mg to about 400 mg twice daily. In another aspect, capivasertib is administered under an intermittent dosing schedule at a dosage from about 400 mg to about 500 mg twice daily. In another aspect, capivasertib is administered under an intermittent dosing schedule at a dosage from about 500 mg to about 600 mg twice daily. In another aspect, capivasertib is administered under an intermittent dosing schedule at a dosage from about 600 mg to about 700 mg twice daily. In another aspect, capivasertib is administered under an intermittent dosing schedule at a dosage from about 700 mg to about 800 mg twice daily. In another aspect, capivasertib is administered under an intermittent dosing schedule at a dosage of about 160 mg twice daily. In another aspect, capivasertib is administered under an intermittent dosing schedule at a dosage of about 200 mg twice daily. In another aspect, capivasertib is administered under an intermittent dosing schedule at a dosage of about 240 mg twice daily. In another aspect, capivasertib is administered under an intermittent dosing schedule at a dosage of about 280 mg twice daily. In another aspect, capivasertib is administered under an intermittent dosing schedule at a dosage of about 320 mg twice daily. In another aspect, capivasertib is administered under an intermittent dosing schedule at a dosage of about 360 mg twice daily. In another aspect, capivasertib is administered under an intermittent dosing schedule at a dosage of about 400 mg twice daily. In another aspect, capivasertib is administered under an intermittent dosing schedule at a dosage of about 440 mg twice daily. In another aspect, capivasertib is administered under an intermittent dosing schedule at a dosage of about 480 mg twice daily. In another aspect, capivasertib is administered under an intermittent dosing schedule at a dosage of about 520 mg twice daily. In another aspect, capivasertib is administered under an intermittent dosing schedule at a dosage of about 580 mg twice daily. In another aspect, capivasertib is administered under an intermittent dosing schedule at a dosage of about 600 mg twice daily. In another aspect, capivasertib is administered under an intermittent dosing schedule at a dosage of about 640 mg twice daily. In another aspect, capivasertib is administered under an intermittent dosing schedule at a dosage of about 680 mg twice daily. In another aspect, capivasertib is administered under an intermittent dosing schedule at a dosage of about 720 mg twice daily. In another aspect, capivasertib is administered under an intermittent dosing schedule at a dosage of about 760 mg twice daily. In another aspect, capivasertib is administered under an intermittent dosing schedule at a dosage of about 800 mg twice daily.

When given as monotherapy, venetoclax can be dosed continuously, e.g., on seven days of a seven-day dosage cycle. For example, from 20 mg to 400 mg of venetoclax can be dosed once daily on a seven-day dosage cycle. In some embodiments, 400 mg of venetoclax can be dosed once daily on a seven-day dosage cycle. In some embodiments, an increasing dosage of venetoclax is given over several dosage cycles (i.e., a ramp-up dosing schedule) to a recommended daily dose. The recommended daily dose can be 400 mg, and lower doses used in monotherapy only as part of a ramp-up schedule. The ramp up schedule can be 20 mg daily during week 1, 50 mg daily during week 2, 100 mg daily during week 3, 200 mg daily during week 4, and 400 mg daily during week 5 and beyond. Additional information can be found in approved labelling for venetoclax.

When given in combination with venetoclax, capivasertib can be dosed intermittently, e.g., on four days of a seven-day dosage cycle, or on two days of a seven-day dosage cycle. For example, from 50 to 900 mg of capivasertib can be dosed twice daily on days one, two, three and four of the first seven-day dosage cycle. In some embodiments, from 300 to 800 mg of capivasertib can be dosed twice daily on days one, two, three and four of the first seven-day dosage cycle. In some embodiments, from 300 to 800 mg of capivasertib can be dosed twice daily on days one and two of a seven-day dosage cycle. In some embodiments, capivasertib is dosed for three weeks but not during week four. Other dosage schedules and dosage amounts can be used.

When given in combination with capivasertib, in some embodiments, venetoclax can be dosed continuously, e.g., on seven days of a seven-day dosage cycle. For example, from 20 mg to 400 mg of venetoclax can be dosed once daily on a seven-day dosage cycle. In some embodiments, 400 mg of venetoclax can be dosed once daily on a seven-day dosage cycle. In some embodiments, an increasing dosage of venetoclax is given over several dosage cycles (i.e., a ramp-up dosing schedule) up to a recommended daily dose of 400 mg.

When given in combination with capivasertib, in some embodiments, venetoclax can be dosed intermittently, e.g., on six days of a seven-day dosage cycle, on five days of a seven-day dosage cycle, on four days of a seven-day dosage cycle, on three days of a seven-day dosage cycle, on two days of a seven-day dosage cycle, or on one day of a seven-day dosage cycle. In some embodiments, doses of venetoclax can be given on days when capivasertib is also given; in other embodiments, doses of venetoclax can be given on days when capivasertib is not given.

On days when both capivasertib and venetoclax are given, the two agents can be dosed together or separately. Being dosed together can be in the form of a fixed-dose combination (e.g., a single tablet or capsule with both agents), or can be in the form of separate dosages given at substantially the same time, i.e., with less than a one-hour delay between dosing the two agents (simultaneous dosing). In some embodiments, venetoclax can be dosed at least two hours after capivasertib or at least four hours after capivasertib. In other embodiments, capivasertib can be dosed at least two hours after venetoclax or at least four hours after venetoclax.

In some embodiments, capivasertib can be dosed on four days of a seven-day dosage cycle, and venetoclax can be dosed on four days of a seven-day dosage cycle. In some embodiments, both agents are given on the same four days, e.g., days one, two, three, and four of a seven-day dosage cycle. In some embodiments, the two agents are not given on the same four days, but are given on different days; e.g., capivasertib on days one, two, three, and four; and venetoclax on days four, five, six, and seven of a seven-day dosage cycle. Other variations are possible.

In some embodiments, capivasertib can be dosed on two days of a seven-day dosage cycle, and venetoclax can be dosed on four days of a seven-day dosage cycle. In some embodiments, both agents are given on overlapping days e.g., days one and two of a seven-day dosage cycle. In some embodiments, the two agents are not given on the same days, but are given on different days; e.g., capivasertib on days one and two; and venetoclax on days three, four, five, and six of a seven-day dosage cycle. Other variations are possible.

In some embodiments, capivasertib can be dosed on four days of a seven-day dosage cycle, and venetoclax can be dosed on three days of a seven-day dosage cycle. In some embodiments, both agents are given on the same days, e.g., capivasertib on days one, two, three, and four, and venetoclax on days one, two and three of a seven-day dosage cycle. In some embodiments, the two agents are not given on the same days, but are given on different days; e.g., capivasertib on days one, two, three, and four; and venetoclax on days five, six, and seven of a seven-day dosage cycle. Other variations are possible.

In some embodiments, capivasertib can be dosed on two days of a seven-day dosage cycle, and venetoclax can be dosed on two days of a seven-day dosage cycle. In some embodiments, both agents are given on the same two days e.g., days one and two of a seven-day dosage cycle. In some embodiments, the two agents are not given on the same days, but are given on different days; e.g., capivasertib on days one and two; and venetoclax on days three and four of a seven-day dosage cycle. Other variations are possible.

In some embodiments, capivasertib can be dosed on four days of a seven-day dosage cycle, and venetoclax can be dosed on two days of a seven-day dosage cycle. In some embodiments, both agents are given on the same days, e.g., capivasertib on days one, two, three, and four, and venetoclax on days one and two of a seven-day dosage cycle. In some embodiments, venetoclax can be given on non-consecutive days, e.g., days one and three of a seven-day dosage cycle. Other variations are possible.

In some embodiments, capivasertib can be dosed on two days of a seven-day dosage cycle, and venetoclax can be dosed on two days of a seven-day dosage cycle. In some embodiments, both agents are given on overlapping days e.g., capivasertib on days one and two of a seven-day dosage cycle, and venetoclax on days one and three of a seven-day dosage cycle. Other variations are possible.

In some embodiments, capivasertib can be dosed on four days of a seven-day dosage cycle, and venetoclax can be dosed on one day of a seven day dosage cycle. In some embodiments, both agents are given on the same day (e.g., capivasertib on days one, two, three and four, and venetoclax on day one of a seven-day dosage cycle). In other embodiments, the agents are given on separate days. Other variations are possible.

In some embodiments, capivasertib can be dosed on two days of a seven-day dosage cycle, and venetoclax can be dosed on one day of a seven day dosage cycle. In some embodiments, both agents are given on the same day (e.g., capivasertib on days one and two, and venetoclax on day one of a seven-day dosage cycle). In other embodiments, the agents are given on separate days. Other variations are possible.

In some embodiments, venetoclax can be given at a dose lower than the recommended daily dose of 400 mg. This may be during a ramp-up dosing schedule in accordance with approved labelling, or can be during ongoing treatment after the ramp-up has been completed. For example, an ongoing dose lower than the recommended daily dose of venetoclax can be 20 mg, 50 mg, 100 mg, 150 mg, 200 mg, 250 mg, 300 mg, or 350 mg.

Thus, in some embodiments, from 50 to 900 mg, or from 300 to 800 mg, of capivasertib can be dosed twice daily on days one, two, three and four of a seven-day dosage cycle; and a dose lower than the recommended daily dose of 400 mg of venetoclax can be dosed once daily in a seven-day dosage cycle.

In some embodiments, from 50 to 900 mg, or from 300 to 800 mg, of capivasertib can be dosed twice daily on days one, two, three and four of a seven-day dosage cycle; and a dose lower than the recommended daily dose of 400 mg of venetoclax can be dosed on four days (e.g., on days one, two, three and four) of a seven-day dosage cycle. Other variations are possible.

In some embodiments, from 50 to 900 mg, or from 300 to 800 mg, of capivasertib can be dosed twice daily on days one and two of a seven-day dosage cycle; and a dose lower than the recommended daily dose of 400 mg of venetoclax can be dosed on four days (e.g., on days one, two, three and four) of a seven-day dosage cycle. Other variations are possible.

In some embodiments, from 50 to 900 mg, or from 300 to 800 mg, of capivasertib can be dosed twice daily on days one, two, three and four of a seven-day dosage cycle; and a dose lower than the recommended daily dose of 400 mg of venetoclax can be dosed on two days (e.g., days one and two; or days one and three) of a seven-day dosage cycle. Other variations are possible.

In some embodiments, from 50 to 900 mg, or from 300 to 800 mg, of capivasertib can be dosed twice daily on days one and two of a seven-day dosage cycle; and a dose lower than the recommended daily dose of 400 mg of venetoclax can be dosed on two days (e.g., days one and two; or days one and three) of a seven-day dosage cycle. Other variations are possible.

In some embodiments, from 50 to 900 mg, or from 300 to 800 mg, of capivasertib can be dosed twice daily on days one, two, three and four of a seven-day dosage cycle; and a dose lower than the recommended daily dose of 400 mg of venetoclax can be dosed on one days (e.g., day one) of a seven-day dosage cycle. Other variations are possible.

In some embodiments, from 50 to 900 mg, or from 300 to 800 mg, of capivasertib can be dosed twice daily on days one and two of a seven-day dosage cycle; and a dose lower than the recommended daily dose of 400 mg of venetoclax can be dosed on one days (e.g., day one) of a seven-day dosage cycle. Other variations are possible.

In some embodiments, treatment with venetoclax begins with a ramp up schedule, whereby the patient takes a low dose in the first dosage cycle (e.g., a seven-day dosage cycle), and progressively larger doses in following dosage cycles until reaching a recommended daily dose (e.g., 400 mg). The ramp up schedule can be 20 mg daily during week 1, 50 mg daily during week 2, 100 mg daily during week 3, 200 mg daily during week 4, and 400 mg daily during week 5 and beyond. Additional information can be found in approved labelling for venetoclax. When given in combination with capivasertib, the ramp up schedule can begin prior to initiation of capivasertib treatment; in conjunction with initiation of capivasertib treatment; or after initiation of capivasertib treatment.

In some embodiments, an initial target regimen includes a target dosage amount and schedule for each of capivasertib and venetoclax, respectively. The initial target regimen may be selected by a clinician and customized to an individual patient's needs. In some embodiments, the initial target regimen includes from 50 to 900 mg, or from 300 to 800 mg, of capivasertib dosed twice daily on days one, two, three and four of a seven-day dosage cycle; and 400 mg of venetoclax dosed daily in a seven-day dosage cycle. In some embodiments, the initial target regimen includes 480 mg of capivasertib dosed twice daily on days one, two, three and four of a seven-day dosage cycle; and 400 mg of venetoclax dosed daily in a seven-day dosage cycle. In some embodiments, the initial target regimen includes 400 mg of capivasertib dosed twice daily on days one, two, three and four of a seven-day dosage cycle; and 400 mg of venetoclax dosed daily in a seven-day dosage cycle. In some embodiments, the initial target regimen includes 640 mg of capivasertib dosed twice daily on days one and two of a seven-day dosage cycle; and 400 mg of venetoclax dosed daily in a seven-day dosage cycle.

In some cases, a patient being treated with an initial target regimen may experience deleterious side effects, including, e.g., body weight loss or undesired changes in blood sugar levels. In such a case where deleterious side effects are in fact observed at the initial target regimen, the initial target regimen can be modified. The modified regimen can be selected so as to mitigate or abrogate deleterious side effects while maintaining a high degree of efficacy. In some embodiments, the initial target regimen is altered with respect to the dosage amount of capivasertib, the dosage schedule of capivasertib, or both, without altering the dosage amount or dosage schedule of venetoclax. In some embodiments, the initial target regimen is altered with respect to the dosage amount of venetoclax, the dosage schedule of venetoclax, or both, without altering the dosage amount or dosage schedule of capivasertib. In some embodiments, the initial target regimen is altered with respect to the dosage amount of venetoclax and/or the dosage schedule of venetoclax, and the dosage amount of capivasertib and/or the dosage schedule of capivasertib.

In some embodiments, a modified regimen involving a reduced dosage amount of venetoclax, reduced dosage frequency of venetoclax, or both, without changes to the dosage amount or dosage schedule of capivasertib, can mitigate or abrogate deleterious side effects experienced with an initial target regiment, while maintaining a high degree of efficacy.

In some embodiments, modifications to the initial target regimen can include changes to the dosage schedule of venetoclax. For example, the dosage schedule can be changed from daily dosing of venetoclax to dosing on six days of a seven-day dosage cycle; on five days of a seven-day dosage cycle; on four days of a seven-day dosage cycle; on three days of a seven-day dosage cycle; on two days of a seven-day dosage cycle; or on one day of a seven-day dosage cycle.

In some embodiments, the initial target regimen can involve dosing venetoclax daily in a seven-day dosage cycle, and the modified regimen can involve dosing venetoclax on four days of a seven-day dosage cycle. The modified regimen can involve dosing capivasertib on four days, or on two days, of a seven-day dosage cycle. The modified regimen can involve dosing capivasertib on days one, two, three, and four of a seven-day dosage cycle, and dosing venetoclax on days one, two, three, and four of a seven-day dosage cycle. The modified regimen can involve dosing capivasertib on days one and two of a seven-day dosage cycle, and dosing venetoclax on days one, two, three, and four of a seven-day dosage cycle.

In some embodiments, the initial target regimen can involve dosing venetoclax daily in a seven-day dosage cycle, and the modified regimen can involve dosing venetoclax on two days of a seven-day dosage cycle. The modified regimen can involve dosing capivasertib on four days, or on two days, of a seven-day dosage cycle. The modified regimen can involve dosing capivasertib on days one, two, three, and four of a seven-day dosage cycle, and dosing venetoclax on days one and two, or days one and three, of a seven-day dosage cycle. The modified regimen can involve dosing capivasertib on days one and two of a seven-day dosage cycle, and dosing venetoclax on days one and two, or days one and three, of a seven-day dosage cycle.

In some embodiments, the initial target regimen can involve dosing venetoclax daily in a seven-day dosage cycle, and the modified regimen can involve dosing venetoclax on one day of a seven-day dosage cycle. The modified regimen can involve dosing capivasertib on four days, or on two days, of a seven-day dosage cycle. The modified regimen can involve dosing capivasertib on days one, two, three, and four of a seven-day dosage cycle, and dosing venetoclax on day one of a seven-day dosage cycle. The modified regimen can involve dosing capivasertib on days one and two of a seven-day dosage cycle, and dosing venetoclax on day one of a seven-day dosage cycle.

In some embodiments, modifications to the initial target regimen can include changes to the dosage amount of venetoclax. For example, the dosage amount can be changed from dosing 400 mg of venetoclax to dosing 350 mg, 300 mg, 250 mg, 200 mg, 150 mg, or 50 mg of venetoclax.

In some embodiments, modifications to the initial target regimen can include changes to both the dosage schedule and the dosage amount of venetoclax. For example, the dosage schedule can be changed from daily dosing of venetoclax to dosing on six days of a seven-day dosage cycle; on five days of a seven-day dosage cycle; on four days of a seven-day dosage cycle; on three days of a seven-day dosage cycle; on two days of a seven-day dosage cycle; or on one day of a seven-day dosage cycle; and the dosage amount can be changed from dosing 400 mg of venetoclax to dosing 350 mg, 300 mg, 250 mg, 200 mg, 150 mg, or 50 mg of venetoclax. Combinations of these changes are contemplated within the scope of the combinations described herein.

The following regimens are illustrative of combination therapies using capivasertib and venetoclax.

### Illustrative Regimen 1

Capivasertib dosed on days one, two, three, and four of a seven-day dosage cycle, 480 mg twice daily, with an optional drug holiday every fourth week; and venetoclax dosed 400 mg daily. Optionally, on days when both are dosed, there is a two or more hour delay between dosing the two agents.

### Illustrative Regimen 2

Capivasertib dosed on days one, two, three, and four of a seven-day dosage cycle, 480 mg twice daily, with an optional drug holiday every fourth week; and venetoclax dosed 400 mg daily on days one, two, three and four of a seven-day dosage cycle. Optionally, on days when both are dosed, there is a two or more hour delay between dosing the two agents.

### Illustrative Regimen 3

Capivasertib dosed on days one, two, three, and four of a seven-day dosage cycle, 480 mg twice daily, with an optional drug holiday every fourth week; and venetoclax dosed 400 mg daily on days one and two of a seven-day dosage cycle. Optionally, on days when both are dosed, there is a two or more hour delay between dosing the two agents.

### Illustrative Regimen 4

Capivasertib dosed on days one, two, three, and four of a seven-day dosage cycle, 480 mg twice daily, with an optional drug holiday every fourth week; and venetoclax dosed 400 mg daily on days one and three of a seven-day dosage cycle. Optionally, on days when both are dosed, there is a two or more hour delay between dosing the two agents.

### Illustrative Regimen 5

Capivasertib dosed on days one, two, three, and four of a seven-day dosage cycle, 480 mg twice daily, with an optional drug holiday every fourth week; and venetoclax dosed 400 mg daily on day one of a seven-day dosage cycle. Optionally, on days when both are dosed, there is a two or more hour delay between dosing the two agents.

### Illustrative Regimen 6

Capivasertib dosed on days one, two, three, and four of a seven-day dosage cycle, 480 mg twice daily, with an optional drug holiday every fourth week; and venetoclax dosed 300 mg daily. Optionally, on days when both are dosed, there is a two or more hour delay between dosing the two agents.

### Illustrative Regimen 7

Capivasertib dosed on days one, two, three, and four of a seven-day dosage cycle, 480 mg twice daily, with an optional drug holiday every fourth week; and venetoclax dosed 200 mg daily. Optionally, on days when both are dosed, there is a two or more hour delay between dosing the two agents.

### Illustrative Regimen 8

Capivasertib dosed on days one, two, three, and four of a seven-day dosage cycle, 480 mg twice daily, with an optional drug holiday every fourth week; and venetoclax dosed 100 mg daily. Optionally, on days when both are dosed, there is a two or more hour delay between dosing the two agents.

### Illustrative Regimen 9

Capivasertib dosed on days one, two, three, and four of a seven-day dosage cycle, 400 mg twice daily, with an optional drug holiday every fourth week; and venetoclax dosed 400 mg daily. Optionally, on days when both are dosed, there is a two or more hour delay between dosing the two agents.

### Illustrative Regimen 10

Capivasertib dosed on days one, two, three, and four of a seven-day dosage cycle, 400 mg twice daily, with an optional drug holiday every fourth week; and venetoclax dosed 400 mg daily on days one, two, three and four of a seven-day dosage cycle. Optionally, on days when both are dosed, there is a two or more hour delay between dosing the two agents.

### Illustrative Regimen 11

Capivasertib dosed on days one, two, three, and four of a seven-day dosage cycle, 400 mg twice daily, with an optional drug holiday every fourth week; and venetoclax dosed 400 mg daily on days one and two of a seven-day dosage cycle. Optionally, on days when both are dosed, there is a two or more hour delay between dosing the two agents.

### Illustrative Regimen 12

Capivasertib dosed on days one, two, three, and four of a seven-day dosage cycle, 400 mg twice daily, with an optional drug holiday every fourth week; and venetoclax dosed 400 mg daily on days one and three of a seven-day dosage cycle. Optionally, on days when both are dosed, there is a two or more hour delay between dosing the two agents.

### Illustrative Regimen 13

Capivasertib dosed on days one, two, three, and four of a seven-day dosage cycle, 400 mg twice daily, with an optional drug holiday every fourth week; and venetoclax dosed 400 mg daily on day one of a seven-day dosage cycle. Optionally, on days when both are dosed, there is a two or more hour delay between dosing the two agents.

### Illustrative Regimen 14

Capivasertib dosed on days one, two, three, and four of a seven-day dosage cycle, 400 mg twice daily, with an optional drug holiday every fourth week; and venetoclax dosed 300 mg daily. Optionally, on days when both are dosed, there is a two or more hour delay between dosing the two agents.

### Illustrative Regimen 15

Capivasertib dosed on days one, two, three, and four of a seven-day dosage cycle, 400 mg twice daily, with an optional drug holiday every fourth week; and venetoclax dosed 200 mg daily. Optionally, on days when both are dosed, there is a two or more hour delay between dosing the two agents.

### Illustrative Regimen 16

Capivasertib dosed on days one, two, three, and four of a seven-day dosage cycle, 400 mg twice daily, with an optional drug holiday every fourth week; and venetoclax dosed 100 mg daily. Optionally, on days when both are dosed, there is a two or more hour delay between dosing the two agents.

### Illustrative Regimen 17

Capivasertib dosed on days one and two of a seven-day dosage cycle, 640 mg twice daily, with an optional drug holiday every fourth week; and venetoclax dosed 400 mg daily. Optionally, on days when both are dosed, there is a two or more hour delay between dosing the two agents.

### Illustrative Regimen 18

Capivasertib dosed on days one and two of a seven-day dosage cycle, 640 mg twice daily, with an optional drug holiday every fourth week; and venetoclax dosed 400 mg on days one, two, three, and four of a seven-day dosage cycle. Optionally, on days when both are dosed, there is a two or more hour delay between dosing the two agents.

### Illustrative Regimen 19

Capivasertib dosed on days one and two of a seven-day dosage cycle, 640 mg twice daily, with an optional drug holiday every fourth week; and venetoclax dosed 400 mg on days one and two of a seven-day dosage cycle. Optionally, on days when both are dosed, there is a two or more hour delay between dosing the two agents.

### Illustrative Regimen 20

Capivasertib dosed on days one and two of a seven-day dosage cycle, 640 mg twice daily, with an optional drug holiday every fourth week; and venetoclax dosed 400 mg on days one and three of a seven-day dosage cycle. Optionally, on days when both are dosed, there is a two or more hour delay between dosing the two agents.

### Illustrative Regimen 21

Capivasertib dosed on days one and two of a seven-day dosage cycle, 640 mg twice daily, with an optional drug holiday every fourth week; and venetoclax dosed 400 mg on day one of a seven-day dosage cycle. Optionally, on days when both are dosed, there is a two or more hour delay between dosing the two agents.

### Illustrative Regimen 22

Capivasertib dosed on days one and two of a seven-day dosage cycle, 640 mg twice daily, with an optional drug holiday every fourth week; and venetoclax dosed 300 mg daily. Optionally, on days when both are dosed, there is a two or more hour delay between dosing the two agents.

### Illustrative Regimen 23

Capivasertib dosed on days one and two of a seven-day dosage cycle, 640 mg twice daily, with an optional drug holiday every fourth week; and venetoclax dosed 200 mg daily. Optionally, on days when both are dosed, there is a two or more hour delay between dosing the two agents.

### Illustrative Regimen 24

Capivasertib dosed on days one and two of a seven-day dosage cycle, 640 mg twice daily, with an optional drug holiday every fourth week; and venetoclax dosed 100 mg daily. Optionally, on days when both are dosed, there is a two or more hour delay between dosing the two agents.

### EXAMPLES

| Abbreviation | Explanation |
|---|---|
| BID | Twice daily |
| BID 10/14 | Twice daily spaced 10 h from AM to PM, and 14 h |
| | PM to AM |
| CR | Complete Response |
| DLBCL | Diffuse Large B Cell Lymphoma |
| DMSO | Dimethyl Sulfoxide |
| GCB | Germinal Center B cell |
| mg/kg | Milligrams per kilogram |
| mL/kg | Milliliters per kilogram |
| PEG | Polyethylene Glycol |
| PO | Per os (oral administration) |
| QD | Daily |
| SCID | Severely combined immunodeficient |
| SEM | Standard Error of the Mean |
| TGI | Tumour Growth Inhibition |

### Example 1—SUDHL4 Human GCB-DLBCL Tumor Cells

For the experiments described below, SUDHL4 human GCB-DLBCL tumor cells (10×10⁶/mouse) were implanted subcutaneously into female CB.17 SCID mice. Mice were randomized into groups of 5 for efficacy and tolerability assessments based on tumor volume and treated with either vehicles (10% DMSO/25% Kleptose pH=5 (Vehicle 1) and 10% ethanol/30% PEG 400/60% Phosal PG-50 (Vehicle 2)), 130 mg/kg or 45 mg/kg capivasertib, 100 mg/kg venetoclax or the combination of capivasertib and venetoclax. Capivasertib was formulated in 10% DMSO/25% Kleptose pH=5 and venetoclax was formulated in 10% ethanol/30% PEG 400/60% Phosal PG-50. All agents were dosed by oral gavage at a 5 ml/kg dose volume according to schedules described, following tumor cell implantation. Tumor length and width was measured by caliper and tumor volume was calculated using the formula (length×width²)*π/6.

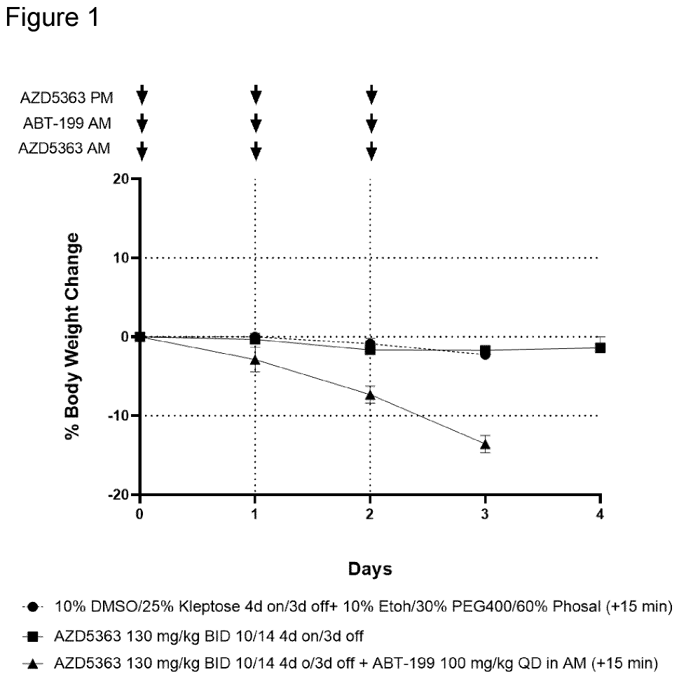

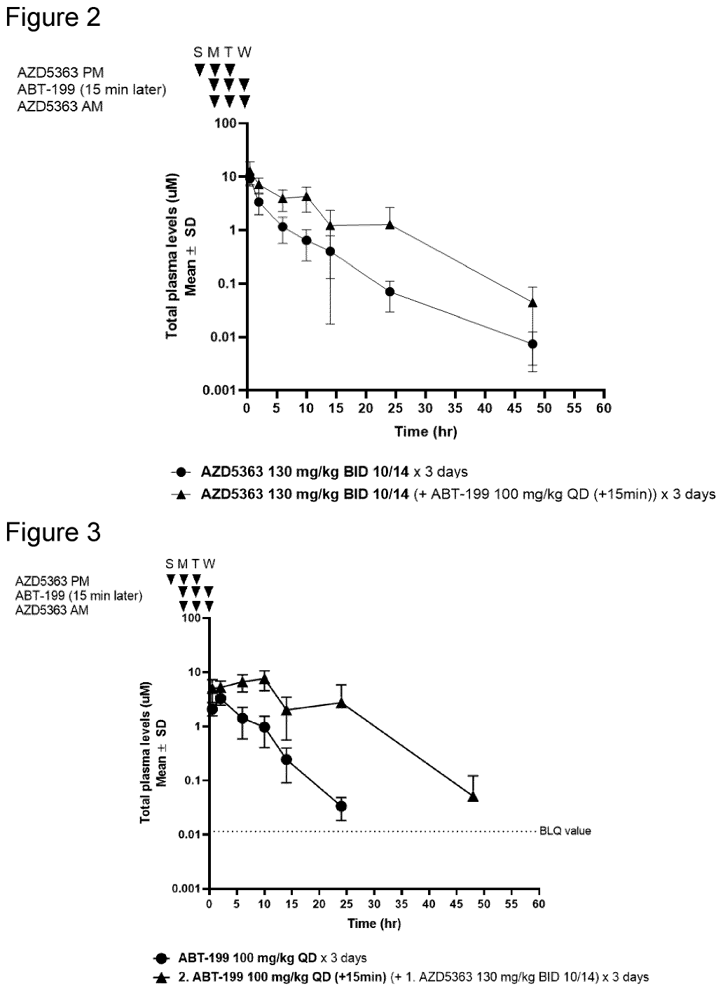

Coadministration of both capivasertib and venetoclax on their respective clinically relevant dose and schedule (capivasertib 130 mg/kg BID 10/14 4 days on/3 days off; venetoclax 100 mg/kg QD in AM (15 min after capivasertib)) resulted in body weight loss (FIG. 1) and drug interactions (FIGS. 2 and 3). This dosing schedule is illustrated in the table below:

| | Day | | | | | | |
|---|---|---|---|---|---|---|---|
| Treatment | 1 | 2 | 3 | 4 | 5 | 6 | 7 |
| capivasertib 130 mg/kg BID 10/14 | X | X | X | X | | | |
| venetoclax 100 mg/kg QD (AM, 15 min | X | X | X | X | X | X | X |
| after capivasertib) | | | | | | | |

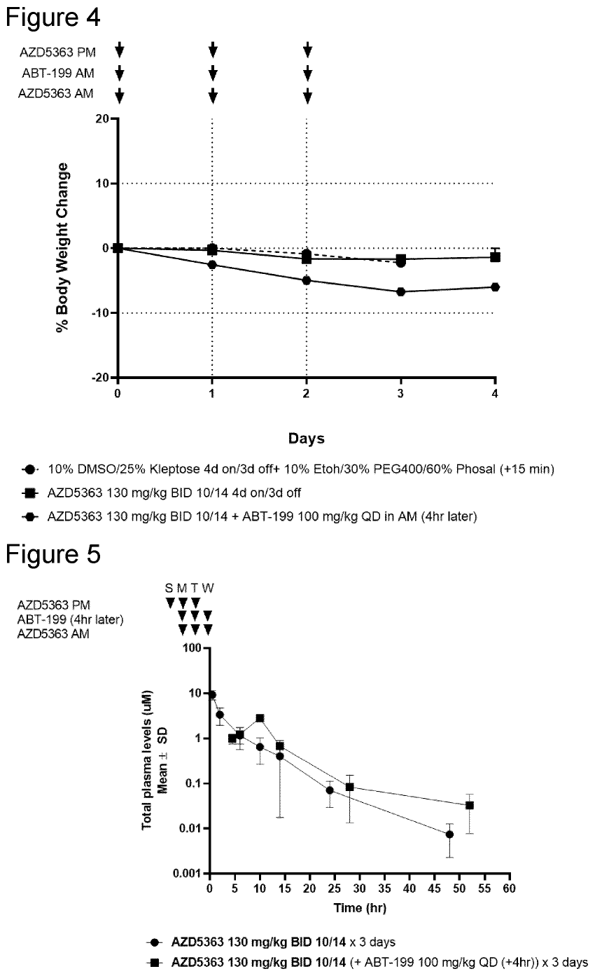

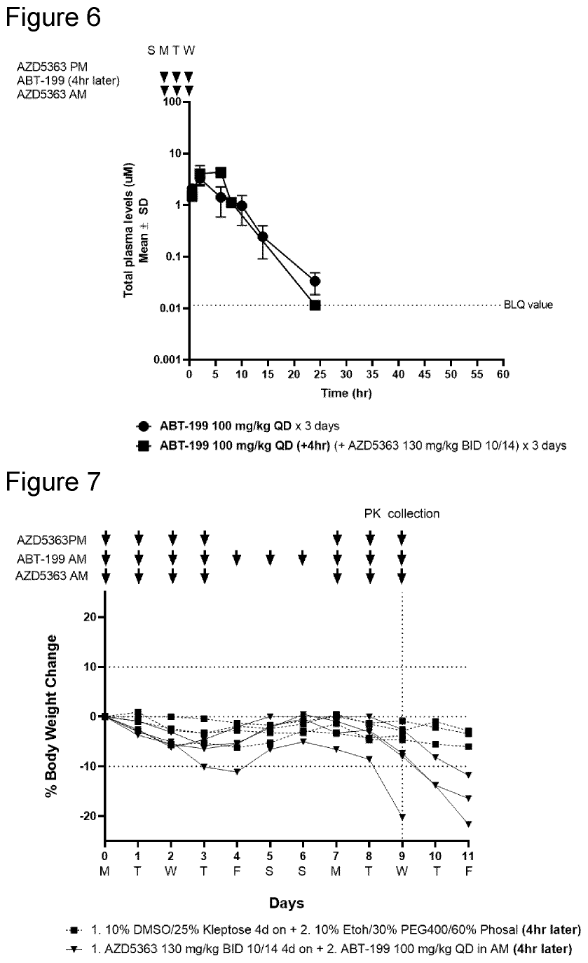

Spacing the dose of capivasertib and venetoclax by 4 hours (capivasertib 130 mg/kg BID 10/14 4 days on/3 days off; venetoclax 100 mg/kg QD in AM (4 hours after capivasertib)) reduced body weight loss (FIG. 4) and improved exposure profiles (FIGS. 5 and 6). However, body weight loss (FIG. 7) and drug interactions were observed after prolonged dosing. This dosing schedule is illustrated in the table below:

| | Day | | | | | | |
|---|---|---|---|---|---|---|---|
| Treatment | 1 | 2 | 3 | 4 | 5 | 6 | 7 |
| capivasertib 130 mg/kg BID 10/14 | X | X | X | X | | | |
| venetoclax 100 mg/kg QD (AM, 4 h | X | X | X | X | X | X | X |
| after capivasertib) | | | | | | | |

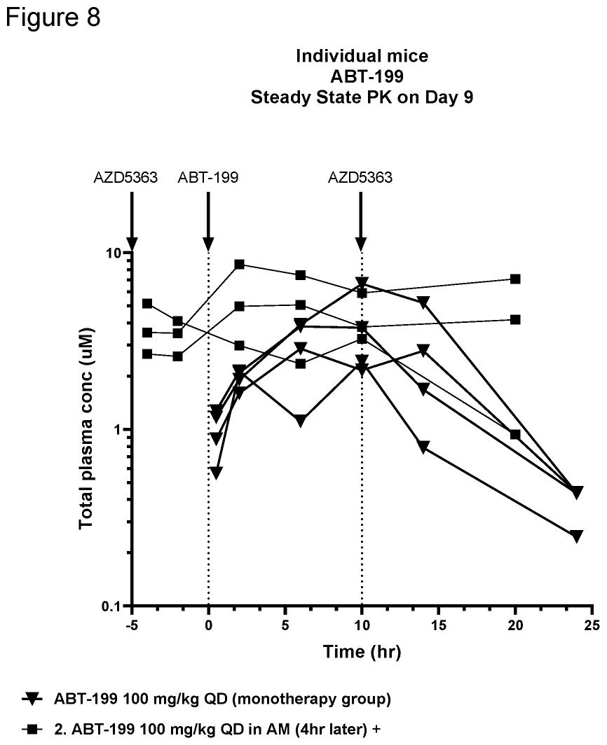

FIG. 8 shows venetoclax plasma concentration following dosing with venetoclax 100 mg/kg QD monotherapy, and capivasertib 130 mg/kg BID 10/14 4 days on/3 days off; venetoclax 100 mg/kg QD in AM (4 hours after capivasertib).

The table below describes further dosing schedules and the resulting TGI and maximum average body weight loss.

| | | | | | | | Max avg body |
|---|---|---|---|---|---|---|---|
| Study | Treatment | Dose | Schedule | TGI | Regression | p-value | weight loss |
| 1 | Vehicle 1 + | 0 mg/kg | BID 10/14 | N/A | N/A | N/A | 2% |
| | Vehicle 2 | | 4 days on/3 days | | | | |
| | | | off × 22 days; QD | | | | |
| | | | 4 days on/3 days | | | | |
| | | | off × 22 days | | | | |
| | Capivasertib | 130 mg/kg | BID 10/14 | 82% | 0% | 0.2904 | 6% |
| | | | 4 days on/3 days | | | | |
| | | | off × 22 days | | | | |
| | Venetoclax | 100 mg/kg | QD (continuous) × | 42% | 0% | 0.003 | 2% |
| | | | 22 days | | | | |
| | Capivasertib + | 130 mg/kg + | BID 10/14 | 100% | 100% | <0.0001 | 13% |
| | Venetoclax | 100 mg/kg | 4 days on/3 days | | | | |
| | | | off × 22 days; QD | | | | |
| | | | in AM (+4 h) | | | | |
| | | | 4 days on/3 days | | | | |
| | | | off × 22 days | | | | |
| | Capivasertib + | 130 mg/kg + | BID 10/14 | 95% | 0% | <0.0001 | 5% |
| | Venetoclax | 100 mg/kg | 4 days on/3 days | | | | |
| | | | off followed by | | | | |
| | | | QD 4 days off/3 | | | | |
| | | | day on × 22 days | | | | |
| | Capivasertib + | 130 mg/kg + | BID 10/14 | 100% | 100% | <0.0001 | 5% |
| | Venetoclax | 100 mg/kg | 4 days on/3 days | | | | |
| | | | off × 22 days; | | | | |
| | | | QW × 22 days | | | | |
| | Capivasertib + | 130 mg/kg + | BID 10/14 | 100% | 100% | <0.0001 | 4% |
| | Venetoclax | 100 mg/kg | 4 days on/3 days | | | | |
| | | | off × 22 days; 2 | | | | |
| | | | QW 22 days | | | | |
| 2 | Vehicle 1 + | 0 mg/kg | BID 10/14 | N/A | N/A | N/A | 6% |
| | Vehicle 2 | | 4 days on/3 days | | | | |
| | | | off × 24 days; QD | | | | |
| | | | 4 days on/3 days | | | | |
| | | | off × 24 days | | | | |
| | Capivasertib + | 45 mg/kg + | BID 10/14 | 36% | 0% | 0.063 | 0 |
| | Venetoclax | 100 mg/kg | 4 days on/3 days | | | | |
| | | | off × 24 days; QD | | | | |
| | | | 5 days on/2 days | | | | |
| | | | off × 24 days | | | | |
| | Capivasertib + | 130 mg/kg + | BID 10/14 | 100% | 100% | <0.0001 | 8% |
| | Venetoclax | 30 mg/kg | 4 days on/3 days | | | | |
| | | | off × 24 days; | | | | |
| | | | QD 5 days on/2 | | | | |
| | | | days off × 24 days | | | | |

In Study 1, twenty-two days of treatment with capivasertib monotherapy resulted in 82% TGI, and venetoclax monotherapy resulted in 42% TGI. When both agents are administered on a simultaneous, 4-day on, 3-day off schedule, 100% tumor regression (5/5 CR) was achieved although with significant body weight loss of 13%. The dosing schedule is illustrated in the table below:

| | Day | | | | | | |
|---|---|---|---|---|---|---|---|
| Treatment | 1 | 2 | 3 | 4 | 5 | 6 | 7 |

| | | | | |
|---|---|---|---|---|
| capivasertib 130 mg/kg BID 10/14 | X | X | X | X |
| venetoclax 100 mg/kg QD (AM, 4 h | X | X | X | X |
| after capivasertib) | | | | |

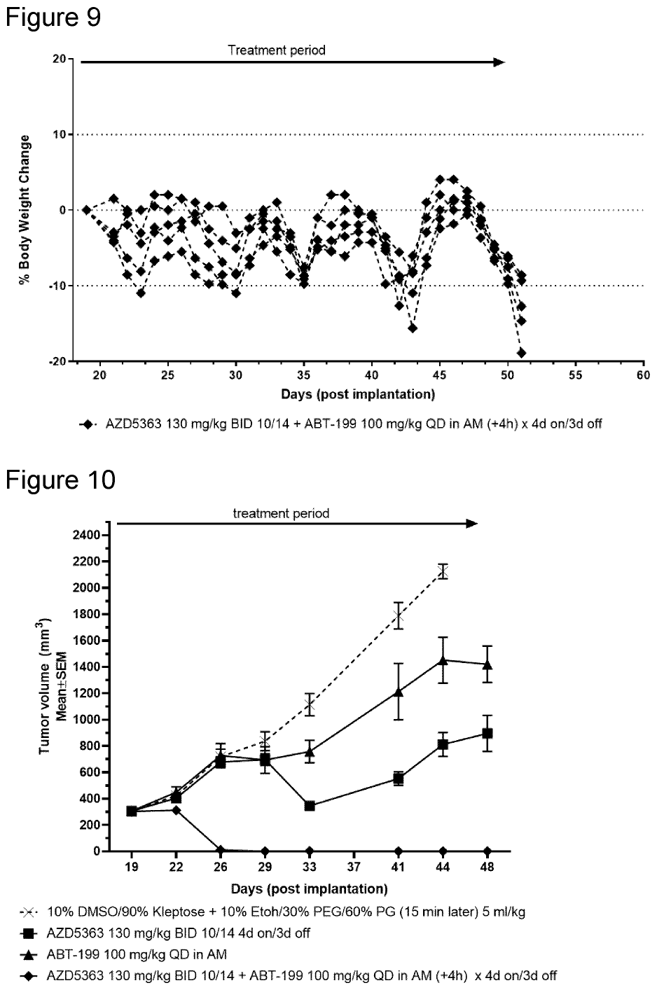

FIG. 9 shows the resulting body weight change over time. FIG. 10 shows the tumor volume over time for both agents (capivasertib BID 10/14 4 days on/3days off×22 days; venetoclax QD in AM (4 hours after capivasertib) 4 days on/3 days off×22 days) vs vehicles and individual drugs dosed on the same schedule. Body weight loss could be controlled by introducing dosing holidays.

In an effort to minimize body weight loss, alternative dosing regimens were examined. Sequencing the dosing of capivasertib and venetoclax on separate days led to 95% TGI with maximal average body weight loss of 5%. The dosing schedule is illustrated in the table below:

| | Day | | | | | | |
|---|---|---|---|---|---|---|---|
| Treatment | 1 | 2 | 3 | 4 | 5 | 6 | 7 |
| capivasertib 130 mg/kg BID 10/14 | X | X | X | X | | | |
| venetoclax 100 mg/kg QD | | | | | X | X | X |

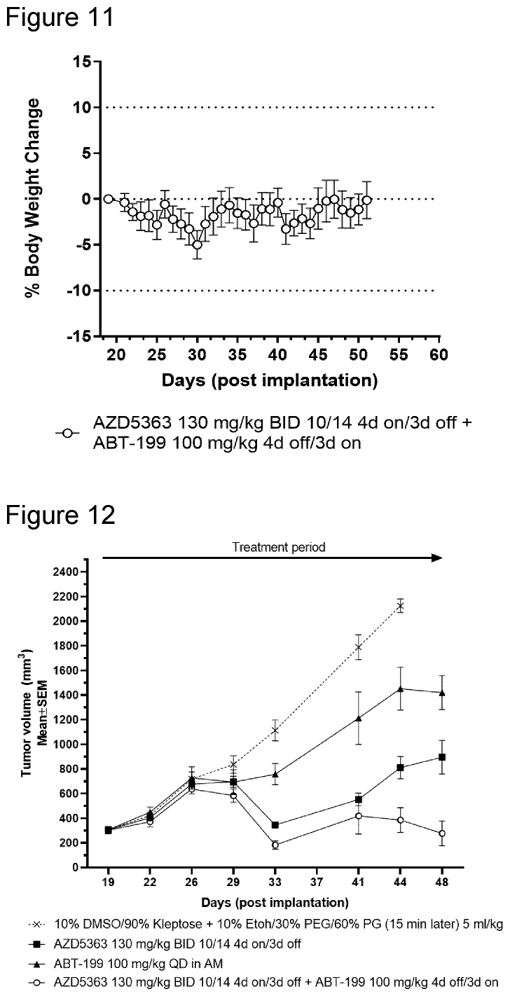

FIG. 11 shows the resulting body weight change over time. FIG. 12 shows the tumor volume over time for both agents on the sequential dosing schedule (capivasertib BID 10/14 4 days on/3days off, venetoclax QD 4 days off/3 days on) vs vehicles and individual drugs.

Reducing the frequency of venetoclax to either once or twice per week in combination with clinically relevant dose and schedule of capivasertib led to 100% tumour regression with minimal body weight loss of ≤5% for either regimen. The dosing schedules are illustrated in the tables below:

| | Day | | | | | | |
|---|---|---|---|---|---|---|---|
| Treatment | 1 | 2 | 3 | 4 | 5 | 6 | 7 |

| | | | | |
|---|---|---|---|---|
| capivasertib 130 mg/kg BID 10/14 | X | X | X | X |
| venetoclax 100 mg/kg 2QW (AM, 4 h | X | | X | |
| after capivasertib) | | | | |

| | Day | | | | | | |
|---|---|---|---|---|---|---|---|
| Treatment | 1 | 2 | 3 | 4 | 5 | 6 | 7 |

| | | | | |
|---|---|---|---|---|
| capivasertib 130 mg/kg BID 10/14 | X | X | X | X |
| venetoclax 100 mg/kg QW (AM, 4 h | X | | | |
| after capivasertib) | | | | |

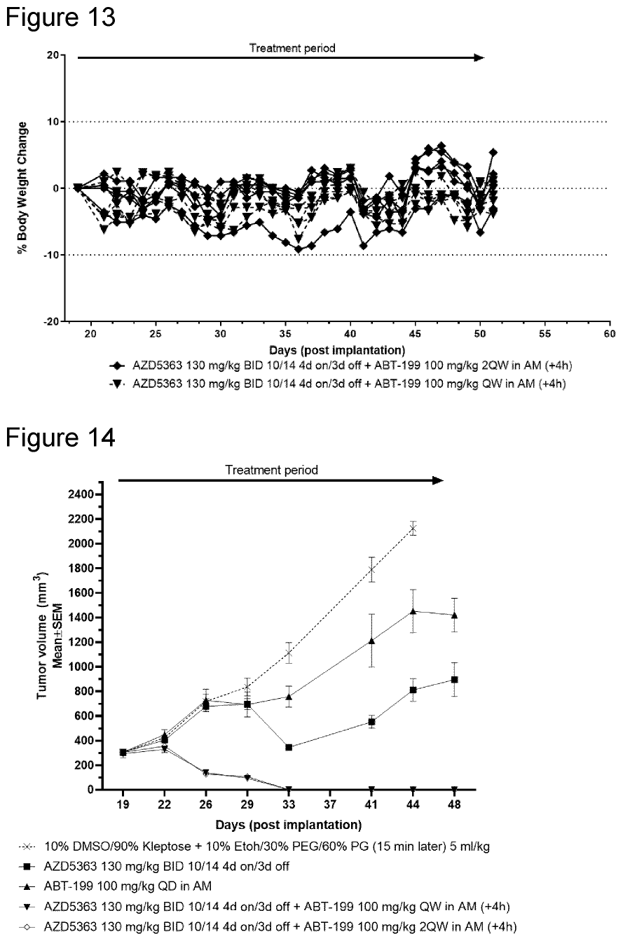

FIG. 13 shows the resulting body weight change over time. FIG. 14 shows the tumor volume over time for both agents (capivasertib BID 10/14 4 days on/3days off; venetoclax QW or 2QW) vs vehicles and individual drugs.

In Study 2, twenty-eight days of capivasertib at 45 mg/kg BID in combination with clinically-relevant dose of venetoclax reduces efficacy in the SUDHL4 xenograft model resulting in 36% TGI. This dosing schedule is illustrated in the table below:

| | Day | | | | | | |
|---|---|---|---|---|---|---|---|
| Treatment | 1 | 2 | 3 | 4 | 5 | 6 | 7 |

| | | | | | |
|---|---|---|---|---|---|
| capivasertib 45 mg/kg BID 10/14 | X | X | X | X | |
| venetoclax 100 mg/kg QD (AM, 15 min | X | X | X | X | X |
| after capivasertib) | | | | | |

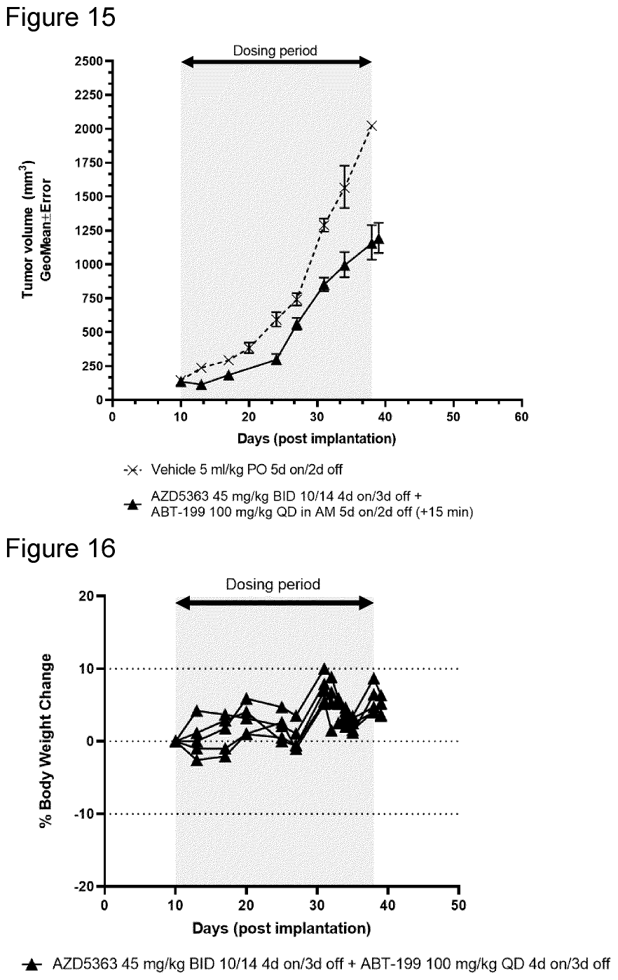

FIG. 15 shows the tumor volume over time vs vehicle. FIG. 16 shows the resulting body weight change over time. The 28-dosing period is indicated in the Figures.

Venetoclax administered at 30 mg/kg QD in combination with clinically relevant dose and schedule of capivasertib was well-tolerated and achieved 100% tumour regressions in the SUDHL4 xenograft model. This dosing schedule is illustrated in the table below:

| | Day | | | | | | |
|---|---|---|---|---|---|---|---|
| Treatment | 1 | 2 | 3 | 4 | 5 | 6 | 7 |

| | | | | | |
|---|---|---|---|---|---|
| capivasertib 130 mg/kg BID 10/14 | X | X | X | X | |
| venetoclax 30 mg/kg QD (AM, 15 min | X | X | X | X | X |
| after capivasertib) | | | | | |

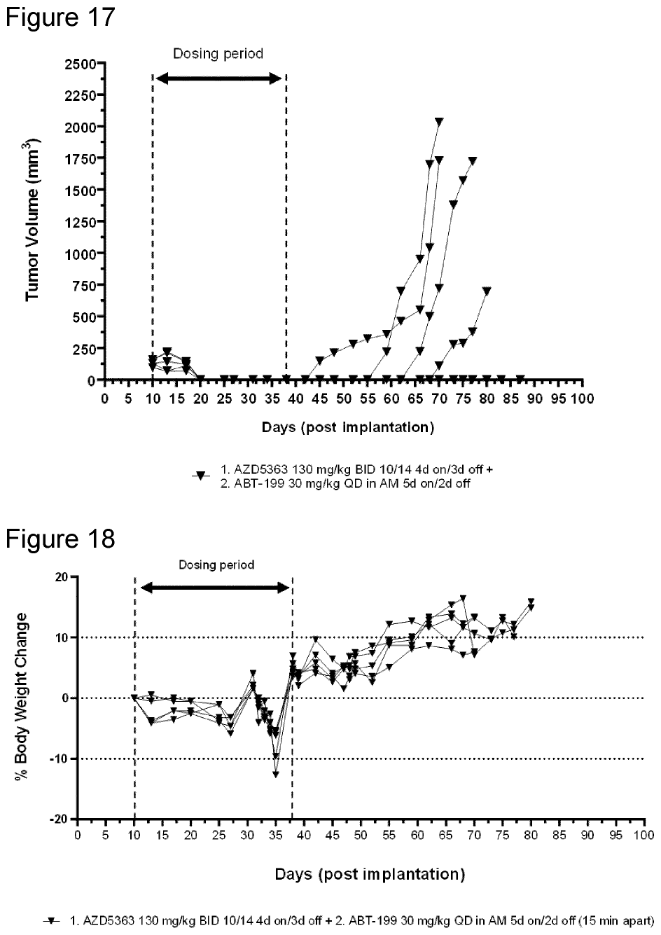

FIG. 17 shows the tumor volume over time vs vehicle. FIG. 18 shows the resulting body weight change over time. The 28-day dosing period is indicated in the Figures.

Other embodiments are within the scope of the following claims.

What is claimed is:

1. A method of treating a B-cell malignancy in a patient in need thereof, comprising: administering to the patient a first amount of capivasertib or a pharmaceutically acceptable salt thereof, and a second amount of venetoclax or a pharmaceutically acceptable salt thereof; wherein the first amount and second amount together comprise a therapeutically effective amount.

2. The method of claim 1, wherein the B-cell malignancy is diffuse large B-cell lymphoma (DLBCL), germinal center B-cell-like diffuse large B-cell lymphoma (GCB-DLBCL), chronic lymphocytic leukemia (CLL), small lymphocytic lymphoma (SLL), or follicular lymphoma.

3. The method of claim 2, wherein the B-cell malignancy is germinal center B-cell-like diffuse large B-cell lymphoma (GCB-DLBCL).

4. The method of any one of claims 1 to 3, wherein the B-cell malignancy is PTEN-null.

5. The method of any one of claims 1 to 3, wherein venetoclax is dosed at least two hours after capivasertib is dosed, on days when both are dosed.

6. The method of any one of claims 1 to 5, wherein capivasertib is dosed on four days in a seven-day dosage cycle.

7. The method of any one of claims 1 to 5, wherein capivasertib is administered at a dose of 300 to 800 mg twice daily, optionally about 320 mg, about 400 mg or about 480 mg twice daily, on days one, two, three and four of a seven-day dosage cycle.

8. The method of any one of claims 1 to 5, wherein capivasertib is dosed on two days in a seven-day dosage cycle.

9. The method of any one of claims 1 to 5, wherein capivasertib is administered at a dose of 300 to 800 mg twice daily, optionally about 480 mg, about 560 mg or about 640 mg twice daily, on days one and two of a seven-day dosage cycle.

10. The method of any one of claims 1 to 9, wherein capivasertib is dosed for three weeks and not dosed during week four.

11. The method of any one of claims 1 to 10, wherein venetoclax is dosed on seven days in a seven-day dosage cycle.

12. The method of any one of claims 1 to 10, wherein venetoclax is dosed one, two, three, four, five, or six days in a seven-day dosage cycle.

13. The method of any one of claims 1 to 10, wherein venetoclax is dosed four days in a seven-day dosage cycle.

14. The method of any one of claims 1 to 10, wherein venetoclax is dosed three days in a seven-day dosage cycle.

15. The method of any one of claims 1 to 10, wherein venetoclax is dosed two days in a seven-day dosage cycle.

16. The method of any one of claims 1 to 10, wherein venetoclax is dosed one day in a seven-day dosage cycle.

17. The method of any one of claims 1 to 10, wherein venetoclax is dosed only on day(s) when capivasertib is dosed.

18. The method of any one of claims 1 to 5 and 10, wherein capivasertib is dosed on two days or four days of a seven-day dosage cycle, and venetoclax is dosed on one, two, three, four, five, six, or seven days of a seven-day dosage cycle.

19. The method of claims 18, wherein venetoclax is dosed on one, two, four, or seven days of a seven-day dosage cycle.

20. A method of treating a B-cell malignancy in a patient in need thereof, comprising:
in a seven-day dosage cycle, administering to the patient:
a first amount of capivasertib or a pharmaceutically acceptable salt thereof on two days or on four days; and
a second amount of venetoclax or a pharmaceutically acceptable salt thereof on seven days; and
in a later seven-day dosage cycle, administering to the patient:
a first amount of capivasertib or a pharmaceutically acceptable salt thereof on two days or on four days; and
a second amount of venetoclax or a pharmaceutically acceptable salt thereof on one, two, three, four, five or six days;
wherein the first amount and second amount together comprise a therapeutically effective amount.

21. The method of claim 20, wherein capivasertib is dosed on days one, two, three and four of a seven-day dosage cycle, and venetoclax is dosed on day one, and on an additional one, two, or three days of the seven-day dosage cycle.

22. The method of claim 20, wherein capivasertib is dosed on days one and two of a seven-day dosage cycle, and venetoclax is dosed on day one, and on an additional one, two, or three days of the seven-day dosage cycle.

23. The method of any one of claims 20 to 22, wherein venetoclax is dosed on day one and either day two or day three of the seven-day dosage cycle.

24. A method of treating a B-cell malignancy in a patient in need thereof, comprising:
in a seven-day dosage cycle, administering to the patient:
a first amount of capivasertib or a pharmaceutically acceptable salt thereof on two days or four days; and
a second amount of venetoclax or a pharmaceutically acceptable salt thereof on seven days; and
in a later seven-day dosage cycle, administering to the patient:
a first amount of capivasertib or a pharmaceutically acceptable salt thereof on two days or four days; and
a third amount of venetoclax or a pharmaceutically acceptable salt thereof on seven days, wherein the third amount is less than the second amount;
wherein the first amount and second amount together comprise a therapeutically effective amount, and wherein the first amount and third amount together comprise a therapeutically effective amount.

25. A method of treating a B-cell malignancy in a patient in need thereof, comprising:
in a seven-day dosage cycle, administering to the patient:
from 300 to 800 mg of capivasertib twice daily on days one, two, and optionally days three and four; and
from 20 to 400 mg of venetoclax once daily on seven days;
in a later seven-day dosage cycle, administering to the patient:
from 300 to 800 mg of capivasertib twice daily on days one, two, and optionally days three and four of the later seven-day dosage cycle; and
from 20 to 400 mg of venetoclax once daily on day one and on an additional one, two, or three days of the later seven-day dosage cycle.

26. Capivasertib for use in the method of any one of the previous claims.

27. Venetoclax for use in the method of any one of claims 1-25.

28. A kit comprising (i) capivasertib, and (ii) instructions for the use of capivasertib in the method of any one of claims 1-25.

29. A combination of capivasertib and venetoclax for use in the method of any one of claims 1-25, wherein the combination is for the simultaneous, separate, or sequential dosing of capivasertib and venetoclax.

30. Use of a combination of capivasertib and venetoclax in the method of any one of claims 1-25, the combination comprising: a first amount of capivasertib or a pharmaceutically acceptable salt thereof, and a second amount of venetoclax or a pharmaceutically acceptable salt thereof, wherein the first amount and second amount together comprise a therapeutically effective amount; wherein the combination is for the simultaneous, separate, or sequential dosing of capivasertib and venetoclax.

31. The combination for use of claim 29 or the use of claim 30, wherein the combination is for separate or sequential dosing.
